(19) United States
(12) Patent Application Publication
(10) Pub. No.: US 2012/0158024 A1
(43) Pub. Date: Jun. 21, 2012

(54) Implant for Treating Gastroesophagael Reflux Disease

(75) Inventor: Justin M. Crank, Maple Grove, MN (US)

(73) Assignee: AMS RESEARCH CORPORATION, Minnetonka, MN (US)

(21) Appl. No.: 13/331,760

(22) Filed: Dec. 20, 2011

Related U.S. Application Data
(60) Provisional application No. 61/425,403, filed on Dec. 21, 2010.

(51) Int. Cl. A61B 17/12

(52) U.S. Cl. 606/151

(57) ABSTRACT

An implant wrapped around the gastroesophageal junction to treat gastroesophageal reflux disease is provided. The implant is positioned around the junction by navigating the implant partly around the junction by a first needle and pulling the remainder of the distance with a second needle. A suture connector can be affixed to the implant with a suture, which can be used to pull the implant into place around the gastroesophageal junction.

### PRIORITY

This application claims priority to and the benefit of U.S. Provisional Patent Application No. 61/425,403, filed Dec. 21, 2010, which is incorporated herein by reference in its entirety.

### FIELD OF THE DISCLOSURE

The present invention is directed to a system for treating gastroesophageal reflux disease and, more particularly, treating gastroesophageal reflux disease by positioning or wrapping an elongated implant or sling to create a cuff around the gastroesophageal junction.

### BACKGROUND OF THE DISCLOSURE

Gastroesophageal reflux disease (“GERD”) is a condition in which the weakening of the lower esophageal sphincter allows stomach acid to enter the esophagus from the stomach. Although, the esophagus comprises a mucus lining protecting the esophagus from the occasional refluxed acid, repeated exposure to refluxed acid can wear away the esophagus lining and permanently damage the walls of the esophagus. As a variety of physiological, psychological and even dietary reasons can cause GERD, selecting the appropriate treatment is difficult and can often limit the effectiveness of any selected treatment. As a result, many treatments for GERD often target the symptoms of GERD rather than the actual causes of GERD.

Implants are an effective treatment option for treating a variety conditions in which a biological fluid flows uncontrollably or with limited control through a biological lumen, such as urinary incontinence. Implantable sling systems, such as the AdVance sling system produced by American Medical Systems of Minnetonka, Minn., comprise anchoring an implantable sling against a biological lumen (e.g., urethra or bladder neck), organ or tissue portion to provide support to that anatomical structure. The sling is typically anchored by navigating the ends of the sling proximate to anchor sites within the patient's body with elongated needles. In order to apply the desired pressure to the target support site, the ends of the sling are anchored to tissue and pulled taut to adjust tension and support characteristics for the particular treatment procedure.

The esophagus is positioned proximate to the rib cage, thereby limiting the available locations where an incision in the patient's body can be made for access to the esophagus, including the lower esophagus. The rib cage limits the direction and angle through which treatment solutions can be inserted and positioned as well. The more awkward the implantation angle required, the greater the likelihood that the vital organs or tissue of the patient will be inadvertently damaged.

### SUMMARY OF THE DISCLOSURE

Embodiments of the present invention are directed to a system for positioning an elongated implant or sling around a gastroesophageal junction, or lower esophagus, of a patient to treat GERD. The system generally comprises a suture advanced partly around the gastroesophageal junction by a first non-linear elongated needle and pulled around the gastroesophageal junction by a second non-linear elongated needle. The suture can be affixed to the end of an implant such that the implant can be pulled around the gastroesophageal junction behind the implantation suture. The implant can then be drawn around the esophageal junction and connected to form a cuff-like configuration to constrict the junction and prevent or limit acid reflux from the stomach.

In this arrangement, the implant can restrict the flow of stomach acid through the esophageal junction by constricting the implant around the esophageal junction. For example, as the stomach becomes distended during a meal, the implant provides resistance to compress the lower esophagus, thereby preventing reflux and providing a valve action for the anatomical structure. Another advantage of the suture approach is that the implant can be positioned around the esophageal junction through a small incision in the abdomen.

The implantation system, according to embodiments of the present invention, generally comprises an implant, an implantation suture, a suture connector, a first elongated needle device and a second elongated needle device. The suture connector can be positioned on the first needle and can comprise a suture mount for receiving the implantation suture. The suture connector can comprise an engagement feature engageable by the second needle for pulling the suture connector off the first needle to draw the implantation suture around the esophageal junction.

To position the implant, the implantation suture can be affixed to the suture mount of the suture connector to operably link the suture connector to the implant. The suture connector is then positioned on the tip of the first needle and advanced through an incision in the patient's body and partly around the esophageal junction. The tip of the second needle is navigated around the esophageal junction from the opposite direction to engage the engagement feature and pull the suture connector off the first needle and around the esophageal junction. The implantation suture can then be pulled to draw the implant around the esophageal junction. The implant can then be tightened, tensioned or otherwise adjusted to provide the desired level of pressure on the esophageal defect. The implantation is severed from the implant and the incision is sutured closed.

According to embodiments of the present invention, the implant can further include a removable casing or sheath for facilitating the navigation of the implant around tissue and the esophageal junction. The removable casing is adapted to receive or shroud the implant and release from the implant once the implant is positioned around the esophageal junction.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE FIGURES

The invention can be completely understood in consideration of the following detailed description of various embodiments of the invention in connection with the accompanying drawings, in which:

FIG. 1 is a representative diagram of the gastroesophageal junction

FIG. 2 shows an implant or sling operably linked to a suture connector in accordance with embodiments of the present invention.

FIGS. 3-4 show elongated needle devices for use in deploying an implant in accordance with embodiments of the present invention.

FIG. 5 shows a slotted end portion of an elongated needle device for use in deploying an implant in accordance with embodiments of the present invention.

FIG. 6 shows an end portion of elongated needle devices for use in deploying an implant having gap portions adapted to selectively engage a connector in accordance with embodiments of the present invention.

FIG. 7 shows a scissor-like elongated needle device for use in deploying an implant in accordance with embodiments of the present invention.

FIGS. 8-9 show a first elongated needle navigating a suture connector at least part of the way around the gastroesophageal junction in accordance with embodiments of the present invention.

FIGS. 10-11 show the transfer of a suture connector from a first elongated needle to a second elongated needle and pulling of the connector and associated implant around the gastroesophageal junction in accordance with embodiments of the present invention.

FIG. 12 shows an implant wrapped around a portion of the gastroesophageal junction to provide treatment for GERD in accordance with embodiments of the present invention.

FIG. 13 shows an implant having at least one grommet or eyelet at an end of the implant to facilitate connecting the implant ends to form a cuff in accordance with embodiments of the present invention.

FIG. 14 shows an implant having an extending suture or filament members, and a deployment sheath, in accordance with embodiments of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

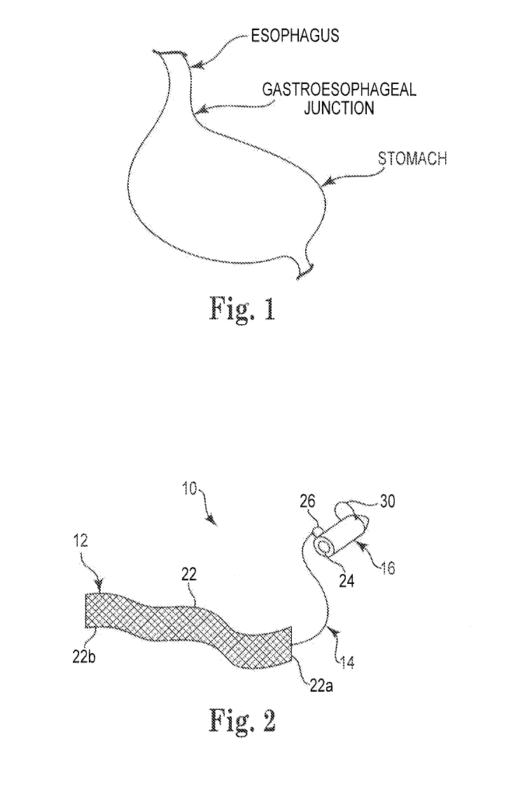

Referring generally to FIGS. 1-14, an implantation system 10 for treating GERD, according to embodiments of the present invention, can comprise an elongated implant or sling 12, an implantation suture 14, a suture connector 16, a first elongated needle device 18 and a second elongated needle device 20. The implant 12 is positionable around an esophageal junction of a patient and adapted to wrap around the junction to apply pressure on, and compress or constrict the flow of fluid through the junction. For example, as the stomach becomes distended during a meal, the implant provides resistance to compress the lower esophagus, thereby preventing reflux and providing a valve action for the anatomical structure.

The implant or sling 12 can include an elongated body portion 22, a first end portion 22a and a second end portion 22b. The implant 12, or parts thereof, can be constructed of a compatible polymer mesh material, or like porous material, known for use and compatibility with urethral slings and other pelvic support devices. Such a mesh embodiment of the implant 12 can facilitate the infiltration of tissue and cells within the implant 12 to promote tissue in-growth and fixation of the device after implantation. The mesh construct can be made of random fibers, woven, braided, twisted or knitted filaments, or formed as a unitary element via molding, laser etching, extrusion, and like processes. Other embodiments of the implant 12 can be non-porous in the form of an elongated thin polymer film or band member.

The suture connector 16 can be connected, directly or indirectly (e.g., via suture 14), to the first end portion 22a of the implant 12 and can include a needle mount or dock feature 24 for releasably engaging with the first non-linear elongated needle 18, and a suture mount 26 for receiving an end of the implantation suture 14. In one embodiment, the suture connector 16 can include an engagement feature 30 engageable by the second non-linear needle 20. The engagement feature 30 can include a suture loop, a mateable latch, a locking ring, a hook or other structures for engaging the tip of a needle so that the second needle 20 is securely engaged with the connector 16 to facilitate manipulation and pulling of the connector 16 and at least a portion of the attached implant 12 around the junction.

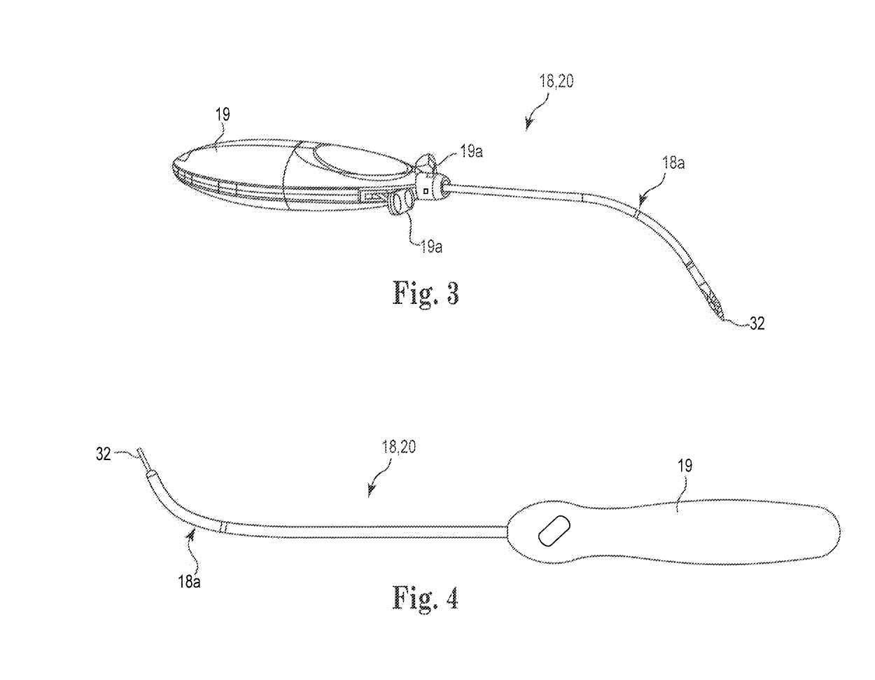

As shown in FIGS. 3-4, the first elongated needle 18 and the second elongated needle 20 can have a handle portion 19 and a needle length 18a that can be non-linear or curved in certain embodiments to facilitate deployment and maneuvering through tissue and around anatomical structure, such as the esophageal junction. In other embodiments, either or both of the needles 18, 20 can be generally straight, helical, or take on a myriad of other shapes and designs to facilitate deployment and use. The handle 19 of either, or both, needle devices 18, 20 can include one or more actuators 19a (slider, button, etc.) operably connected to the needle length 18a to selectively move (retract or extend) a portion of the needle length 18a, as shown in FIG. 3. In such embodiments, the connector 16 can be securely engageable with the distal tip 32 (or 34) such that activation of the one or more actuators 19a selectively disengages the connector 16. Various systems, devices, structures and techniques disclosed in U.S. Patent Application Publication No. 2010/0105979 can be implemented, in whole or in part, with the needle devices 18, 20 of the present invention; therefore, the identified publication is incorporated herein by reference in its entirety.

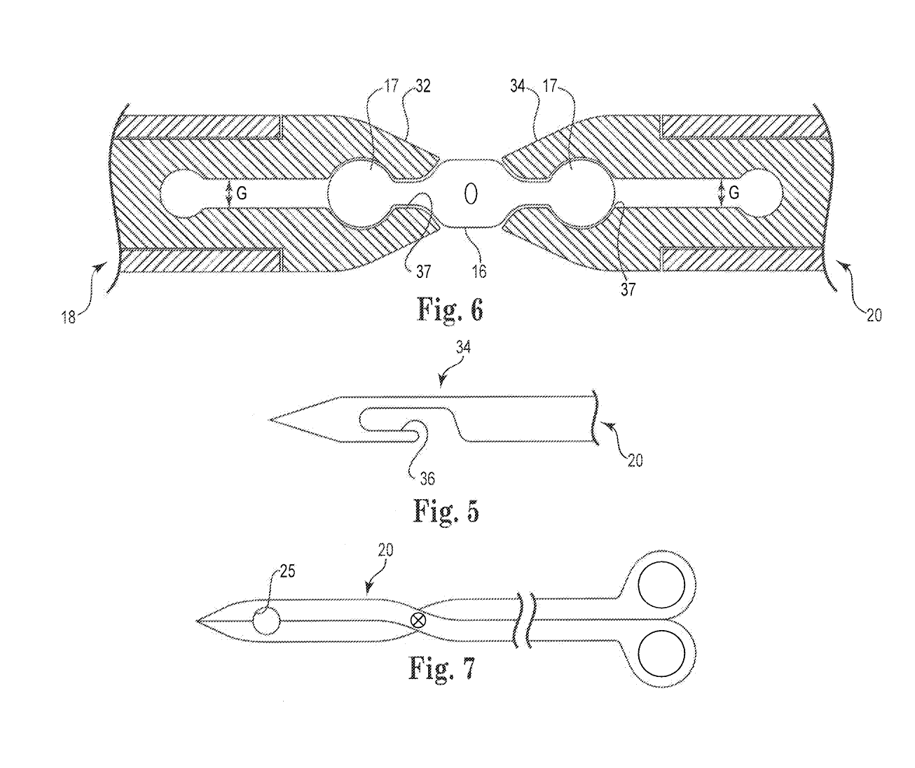

Referring generally to FIGS. 3-8, The first needle 18 includes a needle tip 32 engageable with or receivable by the needle mount or dock portion 24 of the suture connector 16. The second elongated needle 20 also defines a needle tip 34 and can further include a connection feature 36 for releasably engaging the engagement feature 30. The connection feature 36 can be a hook, slot or like construct adapted to releasably catch and attach the engagement feature 30 in certain embodiments, as shown in FIG. 5. In other embodiments, as shown in FIG. 6, each of the elongated needles 18, 20 can include a gap engagement feature 37 having a recess, channel or like opening adapted to receive a portion of the suture connector 16. For example, the suture connector 16 can include one or more bulbous portions 17 to snap, slide or otherwise engage with a like-shaped portion of the engagement feature 37 of one or both of the elongated needles 18, 20. A gap G defines a channel along a portion of the feature 37 to provide a level of selective contraction and expansion of the feature 37 to facilitate retention (e.g., press feature 37 down on portion 17) or release (e.g., expand gap G or reduce pressure of feature 37 on portion 17) of the connector 16 from the respective needle 18, 20. With such a selective engagement configuration, the needles 18, 20 can be easily connected to and disconnected from the connector 16 during various stages of the implantation procedure. FIG. 7 shows another embodiment of the second needle 20 in a scissor-like configuration with an aperture or opening 25 defined therein to permit receipt of the feature 30, such as a suture loop or other like element. To engage the feature 30, the needle 20 can be pivoted open to permit engagement with the feature 30 within the opening 25. The end of the needle 20 can then be closed to trap the feature 30 in the opening for guidance during the procedure, and eventual release upon completion of the deployment or implantation process. Other introducer and deployment devices know to those skilled in the art can be utilized as well without deviating from the scope of the present invention.

The system 10, features, devices, structures and methods detailed herein are envisioned for use with many known pelvic implant and repair systems (e.g., for male and female), features and methods, including those disclosed in U.S. Pat. Nos. 7,500,945; 7,407,480; 7,351,197; 7,347,812; 7,303,525; 7,025,063; 6,691,711; 6,648,921; and 6,612,977, International Patent Publication Nos. WO 2008/057261 and WO 2007/097994, and U.S. Patent Publication Nos. 2011/0144417, 2010/0261955, 2010/0210897, 2010/0105979, 2006/0235262, 2006/0195007, 2002/0151762 and 2002/0147382. Accordingly, the above-identified disclosures are fully incorporated herein by reference in their entirety.

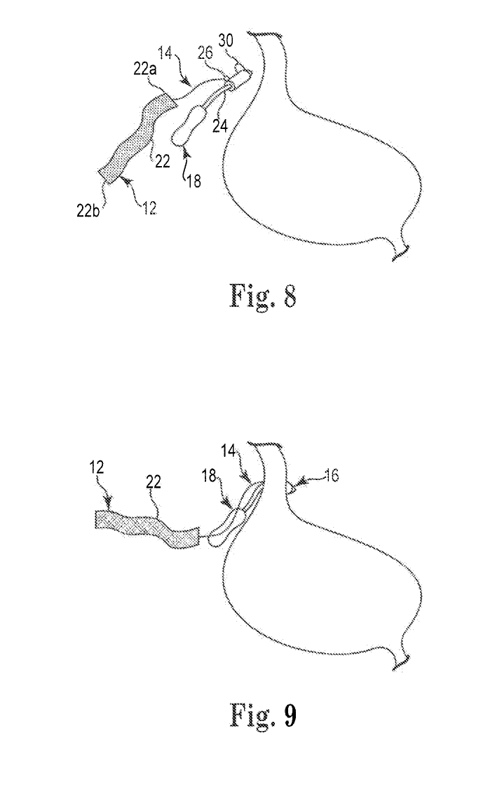

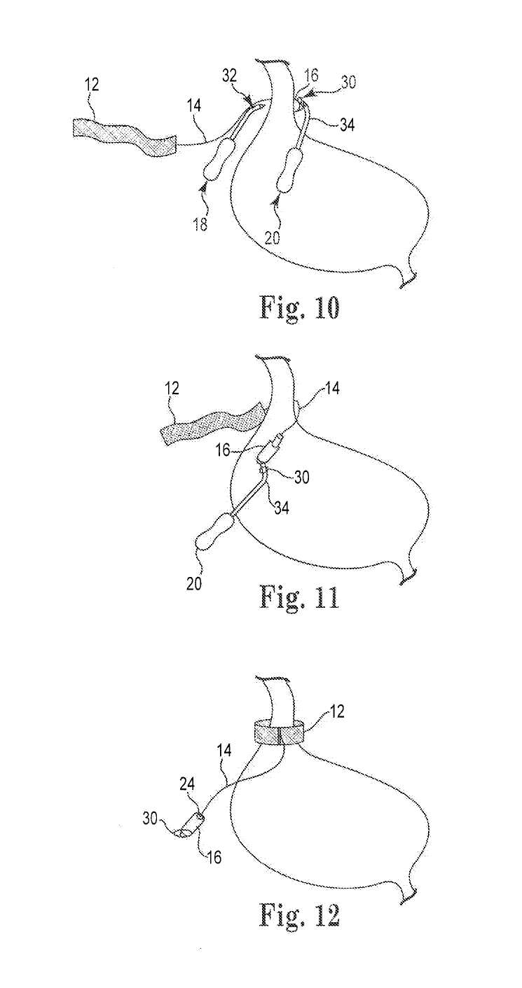

FIGS. 8-12 show embodiments of the present invention in use. The implant 12 of the system 10 can be implanted within the patient's body through an incision in the patient's abdomen, or elsewhere, and directed to an area proximate the esophageal junction as shown in FIG. 3. The implantation suture 14 can be affixed to the first end portion 22a of the implant 12 and the suture mount 26 of the suture connector 16 so as to operably link the implant 12 to the suture connector 16. The dock feature 24 of the suture connector 16 is positioned on the needle tip 32 of the first elongated needle 18. The first elongated needle 18 is adapted to navigate the suture connector 16 and the affixed implantation suture 14 and implant 12 through the incision in the patient and partly around the esophageal junction as shown in FIG. 9. The first needle 18 can include a bent or curved portion to facilitate the navigation of the needle tip 32 through the patient's body and around the esophageal junction, while minimizing the damage to the surrounding tissue. The second elongated needle 20 is adapted to be advanced around the esophageal junction from the opposite side and engage the engagement feature 30 of the suture connector 16 to pull or otherwise remove the connector 16 off the needle tip 32 of the first non-linear needle 18, as shown in FIG. 10. Again, one or more actuators on the first needle 18 can be implemented to assist in releasing the connector 16, or the dock 24, from the tip 32 of the first needle 18. The second needle 20 can similarly include a bent or curved portion to facilitate the navigation of the needle tip 34 around the esophageal junction to engage the engagement feature 30 of the suture connector 16. Once the suture connector 16 is engaged by the second needle 20, the second needle 20 can pull the suture connector 16 and the affixed implantation suture 14 the remainder of the distance around the esophageal junction to pull the implant 12 into position around the esophageal junction, as shown in 11. The implant 12 can then be selectively tightened around the esophageal junction to provide compression and thereby measurably constrict the flow of refluxed acid through the esophageal junction.

Various structures and techniques can be employed with embodiments of the present invention to affix, directly or indirectly, the first end portion 22a and the second end portion 22b of the implant 12 together at this stage of the implant procedure to provide the originally elongated implant 12 as a cuff-like mechanism for compression or restriction on the junction, as shown in FIG. 12. The implantation suture 14 can then be severed or otherwise removed from the implant 12 (e.g., FIG. 12) and the incision in the patient's abdomen can be sutured closed to complete the procedure.

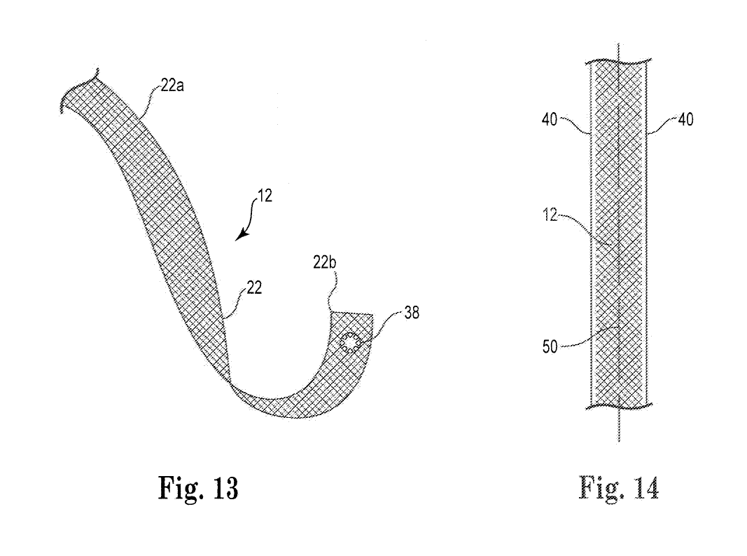

In various embodiments, the first end portion 22a or the second end portion 22b can include a mechanism or construct to facilitate connecting the implant ends in this cuff-like configuration. For instance, as shown in FIG. 13, the second end portion 22b of the implant 12 can include a grommet or eyelet feature 38. The first end portion 22a, or other structures such as the connector 16 or suture 14, can be inserted through the feature 38. In certain embodiments, the grommet feature 38 can include a plurality of angled teeth-like members to catch portions of the inserted implant 12 (e.g., mesh filaments) to secure the insertion portion within the grommet 38 and prevent inadvertent disengagement (e.g., reverse movement out of the grommet). As such, the level of force the wrapped implant 12 exerts around the esophageal junction can be adjusted. In other embodiments, one or both of the implant end portions 22a, 22b can include one or more latches, clips, hooks, extending sutures, anchors, tissue anchors, or like devices or structures to join, link, tie or otherwise attach the end portions 22a, 22b together, directly or indirectly. An extending suture or like filament member 50 is shown in FIG. 14 extending along the longitudinal length of the implant 12. The member 50 can extend a distance past the ends 22a, 22b of the implant such that they can be tied, together or to the opposing implant end portion, upon implantation to join the ends 22a, 22b together to define the junction cuff. The member 50 can be interwoven with or otherwise provided along the implant such that lengths extend from the ends 22a, 22b of the implant 12. In various embodiments, the member 50 extends from one or both of the ends 22a, 22b and does not extend along the entire length of the implant 12.

As shown in FIG. 14, the system 10 can further include a removable casing or sheath 40 for receiving and containing the implant 12 during positioning of the implant 12 around the esophageal junction. The removable casing 40 is generally constructed of a material adapted to pass through the surrounding tissue without undesirably engaging or catching the tissue. The removable casing 40 can be stripped, slid off, or otherwise removed from the implant 12 once the implant 12 is positioned around the esophageal junction, or at other stages of the implantation procedure.

According to embodiments of the present invention, the implantation system 10 can further include one or more ultrasonic transducers for guiding the first needle 18 and/or the second needle 20 through the procedure and around the esophageal junction. The ultrasonic transducer can also be adapted to guide the second needle 20 to engage the suture connector 16 and pull the suture connector 16 off the first needle 18.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

What is claimed:

1. An implant system to compress a body lumen to treat gastroesophageal reflux disease in a patient, comprising:
an introducer device having a handle portion and an elongated needle portion, the elongated needle portion having a distal tip portion;
an elongated implant including a first end portion and a second end portion; and
a connector element having an engagement feature adapted to receive at least the distal tip portion such that the introducer device is adapted to direct the first end portion of the elongated implant around a portion of an esophageal junction of the patient to facilitate compression of the esophageal junction.

2. The system of claim 1, further including a second introducer device having a handle portion and an elongated needle portion, with the elongated needle portion of the second introducer device having a distal tip portion adapted to engage the connector element to further direct the elongated implant around the esophageal junction of the patient.

3. The system of claim 2, wherein the elongated needle portion of the second introducer device is at least partly curved.

4. The system of claim 1, wherein the elongated needle portion of the introducer device is at least partly curved.

5. The system of claim 1, wherein the elongated implant is at least partly constructed of a porous mesh material.

6. The system of claim 1, wherein the engagement feature includes a channel adapted to receive the distal tip portion of the introducer needle device.

7. The system of claim 1, wherein at least the first end portion of the elongated implant includes a fixation element adapted to secure the first end portion of the elongated implant and the second end portion of the elongated implant such that the elongated implant is deployed to securely wrap around the esophageal junction.

8. The system of claim 1, wherein the connector element is provided at the first end portion of the elongated implant.

9. The system of claim 1, further including at least one filament member extending between and connecting the connector element with the first end portion of the elongated implant.

10. The system of claim 9, wherein the at least one filament member is at least one suture.

11. An implant system to treat gastroesophageal reflux disease in a patient, comprising:
a first needle device having a distal tip portion;
a second needle device having a distal tip portion;
an elongated implant including a first end portion and a second end portion; and
a connector element having an engagement feature adapted to receive at least the distal tip portion of the first needle device such that the first needle device is adapted to direct the first end portion of the elongated implant around a portion of an esophageal junction of the patient and the second needle device is adapted to pull the first end portion of the elongated implant the remaining way around the esophageal junction to facilitate compression of the esophageal junction.

12. The system of claim 11, wherein at least the first needle device includes a handle portion having one or more actuators to facilitate engagement between the distal tip portion of the first needle device and the connector element.

13. The system of claim 11, wherein at least a portion of at least one of the first and second needle devices is curved.

14. The system of claim 11, wherein the elongated implant is at least partly constructed of a porous mesh material.

15. The system of claim 11, wherein the engagement feature includes a channel adapted to receive the distal tip portion of the first needle device.

16. The system of claim 11, wherein at least the first end portion of the elongated implant includes a fixation element adapted to secure the first end portion of the elongated implant and the second end portion of the elongated implant such that the elongated implant is securely deployed around a portion of the esophageal junction.

17. The system of claim 16, wherein the fixation element is a grommet element.

18. The system of claim 11, wherein the connector element is provided at the first end portion of the elongated implant.

19. The system of claim 11, further including at least one suture member extending between and connecting the connector element with the first end portion of the elongated implant.

20. The system of claim 11, further including a sheath at least partially shrouding the elongated implant during deployment.
